(19) United States
(12) Patent Application Publication
(10) Pub. No.: US 2018/0200275 A1
(43) Pub. Date: Jul. 19, 2018

(54) METHOD AND THE USE TO IMPROVE ORAL ABSORPTION OF NICOTINAMIDE AND NICOTINAMIDE RIBOSIDE

(71) Applicant: Lawrence Lawrence, Newton, MA (US)

(72) Inventor: Lawrence Lawrence, Newton, MA (US)

(73) Assignee: Better Standing Company Inc., Waban, MA (US)

(21) Appl. No.: 15/839,232

(22) Filed: Dec. 12, 2017

Related U.S. Application Data
(60) Provisional application No. 62/447,018, filed on Jan. 17, 2017.
(60) Provisional application No. 62/450,903, filed on Jan. 26, 2017.
(60) Provisional application No. 62/457,619, filed on Feb. 10, 2017.

(51) Int. Cl. A61K 31/706; A61K 31/352; A61K 31/14; A61K 38/00; A23L 33/15; A61K 9/00; A61K 9/14; A61K 9/48

(52) U.S. Cl. CPC A61K 31/706; A61K 31/352; A61K 31/14; A61K 38/00; A23L 33/15; A23V 2250/7046; A61K 9/148; A61K 9/4875; A23V 2002/00; A23V 2250/2104; A23V 2250/2116; A61K 9/0053

(57) ABSTRACT

The present invention provides methods to improve oral absorption of nicotinamide or nicotinamide riboside or both nicotinamide and nicotinamide riboside via gastric (stomach) uptake or via intestinal uptake into the portal circulation blood. Mixtures of nicotinamide or nicotinamide riboside or both nicotinamide and nicotinamide riboside with botanical sources of flavonoids such as Hibiscus Sabdariffa dried calyces or cocoa powder or aronia berries are prepared. The above mixtures, with a pH modifier if needed, are to make the pyridinium cation form of nicotinamide or nicotinamide riboside or both nicotinamide and nicotinamide riboside and such pyridinium cation forms will form associations of pyridinium cations of nicotinamide or nicotinamide ribose or both vitamins with positively-charged aggregating molecular forms to facilitate transport of the vitamins across gastric wall and intestinal wall mucosal environments to portal blood. This invention discloses different types of above mixtures prepared either for solvation in water solutions or in solid mixtures within gelatin capsules before oral administration prior to solvation in gastric compartment.

### REFERENCE TO RELATED APPLICATIONS

This application claims the benefit of the filing dates of U.S. Provisional Application Ser. No. 62,441,214 filed on Dec. 31, 2016, U.S. Provisional Application Ser. No. 62,447,018 filed on Jan. 17, 2017, U.S. Provisional Application Ser. No. 62,450,903 filed on Jan. 26, 2017, and U.S. Provisional Application Ser. No. 62,457,619 filed on Feb. 10, 2017. The teachings of the entire referenced applications are incorporated herein by reference.

### FIELD OF THE INVENTION

This invention is related to methods to improve oral absorption of nicotinamide or nicotinamide riboside and its applications. More particularly, this invention presents a novel method to accompany the oral absorption of anthocyanins which is a subclass of the flavonoid class or with one or more of any of the subclasses of the flavonoid class which are flavan-3-ols, flavonols, flavanones, flavones, and isoflavones, or any procyanidins or containing any proanthocyanidins or any stilbenoids or any of the foregoing structures which are aglycones or glycosides, with the pyridinium cation of nicotinamide or nicotinamide riboside or with both vitamins to improve their oral absorption into portal circulation blood in a subject.

### BACKGROUND OF THE INVENTION

In the early part of the 20th century, vitamin B3 was identified as a component missing from the diet of pellagra patients. Supplementation with nicotinic acid or niacin, ameliorated the symptoms of pellagra, and prevented the onset of this condition in areas where it was prevalent. The biochemical role of niacin was elucidated in the 1930s, when it was found to be critical for the biosynthesis of nicotinamide adenine dinucleotide (NAD), a compound essential for cellular respiration (Preiss, J.; Handler, P. Biosynthesis of Diphosphopyridine Nucleotide I. Identification of Intermediates J. Biol. Chem. 1958 233, 488-492; Preiss, J.; Handler, P. Biosynthesis of Diphosphopyridine Nucleotide II. Enzymatic Aspects J. Biol. Chem. 1958 233, 493-500). The precise role of NAD in cellular respiration is well understood. As glucose and fatty acids are oxidized, NAD can accept a hydride equivalent, which results in its reduction to NADH. NADH can donate a hydride equivalent, resulting in oxidation back to NAD. These reduction-oxidation cycles use NAD for the temporary storage of hydride ion, but they do not consume NAD. There are other enzymes that use NAD in a different manner, and for purposes not directly related to energy production. Poly-ADPribose polymerases (PARPs), ADPribose transferases (ARTs), and sirtuins all catalyze reactions that release nicotinamide from NAD. This reaction generates a significant amount of energy, similar to ATP hydrolysis. The reverse reaction does not occur readily, so NAD must be replenished by other mechanisms (Bogan, K. L.; Brenner, C. Nicotinic Acid, Nicotinamide, and Nicotinamide Riboside: A Molecular Evaluation of NAD+ Precursor Vitamins in Human Nutrition Annu. Rev. Nutr. 2008, 28, 115-130).

Niacin (or nicotinic acid (pyridine-3-carboxylic acid)), and its amide niacinamide (or nicotinamide (pyridine-3-carboxamide)) are converted to NAD in vivo. In mammals, niacinamide, rather than niacin, is the major NAD precursor. The rate limiting step for this pathway is the formation of the bond between niacinamide and 5-phosphoribose-1-pyrophosphate (PRPP), and it is catalyzed by nicotinamide phosphoribosyl transferase (NAMPT) (Revollo, J. R.; Grimm, A. A.; Imai, S.-I. J. Biol. Chem. 2004, 279, 50754-50763). The NAMPT pathway is thought to be the most efficient route known for nicotinamide recycling.

Nicotinamide, in addition to being known as niacinamide, is also known as 3-pyridinecarboxamide, pyridine-3-carboxamine, nicotinic acid amide, vitamin B3 and vitamin PP. It is represented by the following structural formula:

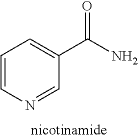

Nicotinamide is one of the two principal forms of the vitamin B3 complex which is commonly used as a collective term to refer to both nicotinamide and nicotinic acid. Nicotinamide and nicotinic acid have identical vitamin recommended daily allowance, but they have very different pharmacological activities. Nicotinamide (niacinamide; nicotinic acid amide, NSA, CAS Nr. 98-92-0) is the amide of nicotinic acid (vitamin B3). It is a water-soluble vitamin and part of the vitamin B group. As such, it fulfills an important function in the human and animal body by virtue of being a part of the coenzymes NAD⁺ and NADP⁺. Nicotinamide deficiency in humans leads to various symptoms, including weight loss, memory disruption, sleep disorders and the skin disease pellagra, all of which can be treated by administering Nicotinamide.

Nicotinamide is used as a food additive and pharmaceutical. It is usually applied as part of mixed compositions with other components, especially other vitamins. A common dosage form of Nicotinamide or compositions comprising Nicotinamide is a tablet.

In 1982, nicotinamide riboside (NR) was investigated as a NAD precursor in prokaryotes (Liu, G.; Foster, J.; Manlapaz-Ramos, R.; Loivera, B. M. “Nucleoside Salvage Pathway for NAD Biosynthesis in Salmonella typhimurium” J. Bacteriol. 1982, 152, 1111-1116). Although NR appears to be a natural precursor for NAD, it likely contributes only a small amount to NAD biosynthesis owing to the apparent scarcity of NR in dietary sources. NR contains a high energy glycosidic bond that is spontaneously labile in aqueous solution, yielding nicotinamide and ribose decomposition products. In rats NR is not taken up by intestinal absorption, rather releasing nicotinamide in intestinal mucosal cells for absorption into portal circulation. (Gross, C. J.; Henderson, L. M. “Digestion and Absorption of NAD by the Small Intestine of the Rat” J. Nutr. 1983, 113, 412-420).

This spontaneous reaction occurs over the course of hours or days depending on the exact ambient conditions, but it makes any naturally occurring NR difficult to keep in food sources, while nicotinic acid or nicotinamide are considerably more stable and easy to prepare and administer. NR has been reported to occur in milk (Bieganowski and Brenner (2004) Cell 117: 495-502) and beer, but the amounts typically present are probably too small to be nutritionally significant.

Currently, NR supplementation is limited by the available commercial supply. NR supplementation could represent a dietary alternative to niacin, with the advantage of being a more efficient NAD precursor. By taking advantage of a natural pathway to synthesize NAD while consuming less energy, NR could offer benefits for human health. Cells are constantly subject to damage by normal environmental factors, and they have evolved repair mechanisms to continuously reverse this damage. The repair mechanisms consume NAD by scission of the high energy glycosidic linkage to produce species such as poly-ADPribose and ADP-ribosylated proteins. In severely damaged cells, energy stores are not sufficient to produce the NAD necessary to maintain homeostasis, and the damage becomes irreversible. Therefore, an energy-rich NAD precursor such as NR may be able to address cell and tissue damage at the molecular level.

Nicotinamide riboside (CAS Number 1341-23-7) is a precursor to nicotinamide adenine dinucleotide (NAD) and represents a source of vitamin B3. Recent studies have indicated that novel health benefits may result from ingesting nicotinamide riboside in larger quantities than is found naturally in foods. For example, nicotinamide riboside has been implicated in raising tissue NAD concentrations and in eliciting insulin sensitivity and enhancement of sirtuin functions. See Chi Y, et al., Curr Opin Clin Nutr Metab Care. 2013 November; 16(6):657-61. Its ability to increase NAD production indicates that nicotinamide riboside can also increase mitochondrial health, stimulate mitochondrial function, and induce creation of new mitochondria. Additional studies with nicotinamide riboside in models of Alzheimer's disease have suggested that the molecule is bioavailable to the brain and provides neuroprotective effects, likely by stimulation of brain NAD synthesis. Furthermore, a 2012 study observed that mice on a high-fat diet that was supplemented with nicotinamide riboside gained 60% less weight than mice eating the same high-fat diet without nicotinamide riboside.

However, the benefits of nicotinamide or nicotinamide ribose or both vitamins are limited by the low uptake efficiency within gastric cell layers, intestinal cell layers, and hepatic cell membranes that are responsible for absorption into portal side circulation blood and for bioavailability to biotransformation enzymes. Thus, there is a need to increase the absorption efficiency.

### SUMMARY OF THE INVENTION

The present invention is to make the pyridinium cation of nicotinamide or nicotinamide riboside or both vitamins, available to cation-π interactions that are binding associations with aromatic systems contained in molecular structures of any of the major subclasses of flavonoids, anthocyanidins (anthocyanins), flavan-3-ols, flavonols, flavanones, flavones, and isoflavones, or any procyanidins or any proanthocyanidins or any stilbenoids or any of the foregoing structures which are aglycones or glycosides, with solution conditions having pH value below pH 4.1 and preferably near pH 3. Nicotiamide riboside used alone in the present invention does not require pH adjustment to form its pyridinium cation.

The present invention is to deliver positively-charged aggregated molecular forms comprising above said amphiphilic molecular structures which spontaneously undergo aggregation in aqueous solution with bound nicotinamide or bound nicotinamide riboside or bound both vitamins and prior to absorption into the portal circulation.

The present invention provides an efficient means of delivering said aggregated forms of bound vitamin(s) to enzymes present in portal circulation blood or within hepatic tissues by utilizing the affinity for cell membrane environments. This invention achieves elevated concentrations of the resulting enzyme products, including but not limited to nicotinamide mononucleotide and N(1)-methylnicotinamide.

### DETAILED DESCRIPTION OF THE PREFERRED EMBODIMENTS

The present invention is to make the pyridinium cation of nicotinamide or nicotinamide riboside or both vitamins, available to cation-π interactions that are binding associations with aromatic systems contained in molecular structures of any of the major subclasses of flavonoids, anthocyanidins (anthocyanins), flavan-3-ols, flavonols, flavanones, flavones, and isoflavones, or any procyanidins or any proanthocyanidins or any stilbenoids or any of the foregoing structures which are aglycones or glycosides.

The present invention is to deliver aggregated molecular forms comprising above said amphiphilic molecular structures with bound nicotinamide or bound nicotinamide riboside or bound both vitamins into the portal circulation. Positively charged aggregates derived from strongly amphiphilic pharmaceutical agents are known to efficiently enter portal circulation via the gastric wall. (Frenkel, Y. V.; Gallicchio, E.; Das, K.; Levy, R. M.; Arnold E. “Molecular dynamics study of non-nucleoside reverse transcriptase inhibitor 4-[[4-[[4-[(E)-2-cyanoethenyl]-2,6-dimethylphenyl]amino]-2 -pyrimidinyl]amino]benzonitrile (TMC278/rilpivirine) aggregates: correlation between amphiphilic properties of the drug and oral bioavailability” Med Chem. 2009, 52, 5896-5905). The flavylium ions of anthocyanins and anthocyanidins are strongly amphiphilic and undergo self-assembly (aggregation) in water solutions. (Pina, F.; Melo, M. J.; Laia, C. A.; Parola, A. J.; Lima, J. C. “Chemistry and applications of flavylium compounds: a handful of colours” Chem. Soc. Rev., 2012, 41, 869-908, see especially page 886). It is evident that in humans and rodents that strongly amphiphilic anthocyanins are rapidly absorbed from stomach into portal blood and circulated without degradation (Passamonti, S.; Vrhovsek, U.; Vanzo, A.; Mattivi F. “The stomach as a site for anthocyanins absorption from food” FEBS Lett. 2003, 544, 210-3). It is expected that acidic environment in stomach facilitates absorption of the flavylium ions in aggregate forms across gastric wall.

The present invention provides an efficient means of delivering said aggregated forms of bound vitamin(s) to enzymes present in portal circulation blood or within hepatic tissues by utilizing the affinity for cell membrane environments. This invention achieves elevated concentrations of the resulting enzyme products, including but not limited to nicotinamide mononucleotide and N(1)-methylnicotinamide. Also, the present invention provides an efficient method of delivering said aggregated forms of bound vitamin(s) to enzymes that reside within hepatocytes or near hepatocyte cell membranes through liver entry via hepatic sinusoid endothelial cell fenestrations.

The present invention provides conditions for dissolving nicotinamide or nicotinamide riboside or both vitamins into aqueous solution with the purpose of engendering said cation-π interactions with any of said molecular structures of said flavonoids or any procyanidins or any proanthrocyanidins or any stilbenoids or any of these structures which are aglycones or glycosides. (“Molecular recognition in aqueous media. New binding studies provide further insights into the cation-.pi. interaction and related phenomena”, Kearney, P. C.; Mizoue, L. S.; Kumpf, R. A.; Forman, J. E.; McCurdy, A.; Dougherty, D. A. J. Am. Chem. Soc., 1993, 115, 9907-9919). The present invention also provides conditions for engendering said cation-π interactions early in the onset of said solvation of a mixture of said vitamins and said molecular structures which are amphiphilic, as are also nicotinamide and nicotinamide riboside.

In order to form pyridinium cation of nicotinamide, the present invention provides solution conditions having pH value below pH 4.1, and preferably near pH 3 by dissolution of a pH modifier such as abundantly present in dried pulverized calyces of hibiscus sabdariffa as hibiscus acid lactone or such as adding citric acid or gluconic acid. The pyridinium cation of nicotinamide predominates in solutions made using the present invention since the pyridine ring nitrogen atom has pKa 4.14 (“Hyperpolarized 15N-pyridine Derivatives as pH-Sensitive MRI Agents”, Jiang, W.; Lumata, L.; Chen, W.; Zhang, S.; Kovacs, Z.; Sherry, A. D.; and Khemtong, C. Sci Rep. 2015, 16; 5:9104). Said associations based on cation-π interactions are expected to be taking place in aqueous solutions coincidently with inter-molecular self-associations that involve molecular forms of said flavonoids, procyanidins, proanthocyanidins, or stilbenoids. Said inter-molecular self-associations lead to colloidal aggregation processes.

Occurrences of said inter-molecular associations are expected to take place rapidly in consequence to exposing amphiphilic (amphipathic) structures to aqueous environment and produce aggregated molecular forms. Engendering cation-π interactions that are binding associations in aqueous solution between said pyridinium cations and aromatic (π-electron) systems of said flavonoids, procyanidins, or proanthrocyanidins or stilbenoids will result in aggregated molecular forms bearing multiple positive charges. The nitrogen of nicotinamide ribose is positively charged in aqueous solvents as consequence of the carbon-nitrogen bond between the ribose ring and pyridinium ring. The present invention is to facilitate said cation-π interactions in order to associate nicotinamide or nicotinamide ribose or both vitamins within aggregated molecular forms and to thereby accomplish their gastric and intestinal absorption with said aggregated molecular forms into portal circulation blood. Conditions in the intestinal and intercellular environment and in the portal blood which subsequently elevate pH from that in the stomach are expected to result in the disappearance over time of the pyridinium cation form of nicotinamide, and consequently the disappearance of cation-π interactions. However, this will not readily result in dissociation of bound nicotinamide from the aggregated molecular forms because of other available noncovalent interactions within the said aggregated forms.

The present invention is to facilitate said cation-π interactions in order to form associations of pyridinium cations of nicotinamide or nicotinamide ribose or both vitamins within aggregated molecular forms and to thereby furnish positively-charged aggregated molecular forms to gastric wall and intestinal wall mucosal environments. Thus, this present invention is to facilitate entry of said aggregated forms with said bound vitamins into the gastric wall and other intestinal cell layers and to thereby avail of absorption processes responsible for the appearance of said aggregated forms with said bound vitamins in the portal circulation blood.

The present invention thereby provides said aggregated forms with bound forms of nicotinamide or nicotinamide ribose or both vitamins to the uptake processes within gastric and intestinal cell layers that are responsible for absorption into portal side circulation blood. This present invention also utilizes the affinity for hepatic cell membranes of charged or uncharged molecular aggregated forms with bound nicotinamide, bound nicotinamide ribose or with bound both vitamins.

The present invention aims to increase the concentration of nicotinamide or nicotinamide riboside or both vitamins in the portal circulation blood. This invention retains nicotinamide riboside using said aggregated forms and thereby lessens interactions in the intestinal environment responsible for biotransformation of nicotinamide riboside to nicotinamide. Also, the present invention aims to increase nicotinamide concentration or nicotinamide riboside concentration or concentrations of both vitamins in hepatic cell membranes and thereby increases the bioavailability of said vitamins in liver hepatocytes. Thus, this invention helps to increase in vivo biosynthesis of nicotinamide mononucleotide, nicotinamide adenine dinucleotide (NAD), and nicotinamide adenine dinucleotide phosphate [NADPH]. It also may help to increase hepatic biotransformation of nicotinamide to N(1)-methylnicotinamide. Conversion of nicotinamide or nicotinamide riboside to nicotinamide mononucleotide retains these vitamins in hepatic and other cells, and thereby limits excretion from the body of these soluble B vitamins.

Nicotinamide can be converted into nicotinamide mononucleotide by intracellular and extracellular forms of nicotinamide phosphoribosyltransferase, the rate-limiting enzyme in the NAD biosynthetic pathway starting from nicotinamide. (Frye, R. A. Biochem. Biophys. Res. Commun. 2000, 273, 793-798)

Nicotinamide can be converted into N(1)-methylnicotinamide by Nicotinamide N-methyltransferase, which is present in mammals principally in the liver. (Pissios P. Nat Med. 2015 21, 887-894) Nicotinamide riboside can be converted into nicotinamide mononucleotide by intracellular NR kinases. (Ratajczak, J.; Joffraud, M.; Trammell, S. A.; Ras, R.; Canela, N.; Boutant, M.; Kulkarni, S. S.; Rodrigues, M.; Redpath, P.; Migaud, M. E.; Auwerx, J.; Yanes, O. Brenner, C.; Cantó, C. “NRK1 controls nicotinamide mononucleotide and nicotinamide riboside metabolism in mammalian cells” Nat Commun. 2016, 11, 7:13103)

Bioavailability to said phosphoribosyltransferase of nicotinamide administered orally in pharmacological (milligram) quantities is limited by absorption of self-assembled aggregated forms of nicotinamide into portal blood circulation and subsequently entry of said self-assembled forms into hepatocyte cell membranes. Low bioavailability of nicotinamide in its self-assembled forms to said phosphoribosyltransferase should be considered responsible for low biotransformation of nicotinamide to nicotinamide mononucleotide before its metabolism and excretion after oral administration of pharmacological (milligram) nicotinamide doses. Contrastingly, nutritive absorption of nicotinamide from dietary intake of foods is expected to occur without self-assembled forms of nicotinamide appearing in portal circulation and hepatitis cell membranes and thus favors interaction of nicotinamide with said phosphoribosyltransferase.

Bioavailability of nicotinamide riboside administered orally is known to be limited by biotransformation of nicotinamide riboside to nicotinamide in the intestinal wall (Gross, C. J.; Henderson, L. M. ‘Digestion and absorption of NAD by the small intestine of the rat’, J Nutr. 1983, 113, 412-420). Nicotinamide riboside concentrations achieved in portal blood circulation and consequently afterward in hepatic tissues are then available to constitutive kinase activity and thus to favor production of nicotinamide mononucleotide by an alternative route to that of nicotinamide which relies on phosphoribosyltransferase activity. However, the phosphoribosyltransferase activity is limited are common in wide-ranging circumstances including aging, sedentary lifestyle, obesity, and other health disorders.

The present invention demonstrated a method of facilitating oral absorption and high bioavailability of said vitamins via gastric (stomach) uptake or via intestinal uptake or via both uptake routes into portal circulation blood and hepatic tissues, using pharmacological (milligram) doses of either nicotinamide (3-pyridine-carboxamide) or nicotinamide riboside (N-ribosylnicotinamide chloride) or both vitamins. High or low (milligram) amounts of these vitamins may be used if desired as long as their binding interaction partners on a mole basis remain in excess, without requiring changes to amounts of the other constituents placed into solutions made for oral administration.

A method of increasing oral absorption via the gastric (stomach) route into the portal circulation was demonstrated. The oral absorption of either 50 mg of nicotinamide (3-pyridine-carboxamide) or 110 mg of nicotinamide riboside (from 125 mg of N-Ribosylnicotinamide chloride) was demonstrated after either as dry powder of nearly 100% purity was mixed into nine (9) grains of pulverized dried calyces of hibiscus sabdariffa flowers (sourced from Frontier Natural Products Co-op https://wholesale.frontiercoop.com/shop/herbal-teas-2/frontier-organic-cut-sifted-hibiscus-flowers-1-1/one/ and processed in a coffee mill) containing an excess of anthocyanins on a molar basis relative to either vitamin and hibiscus acid among other constituents normally present. Steeping either composition with six (6) ounces of either distilled or municipal ‘tap’ water at 95-100° C. was done in an infusion compartment (AeroPress, Aerobie, Inc. Palo Alto, Calif.) with integrated filter paper holder for two (2) minutes before pressurizing the compartment to pass infusion over paper filter into glass. Another four (4) ounces of water at 95-100° C. was added to the solids remaining in compartment and stirred to form new infusion before filtrate was passed into same glass containing first filtrate. Total filtrate volume was approximately seven (7) ounces. The optical depth and deep-red color of the filtrate indicated hibiscus sabdariffa anthocyanins were predominately in their flavylium cation forms as expected from the pH of the filtrate which was tested with pH strips. The strip coloration indicated approximately pH 3, as expected due to presence of hibiscus acid dissolved in the filtrate. A person swallowed the entire volume of filtrate within five (5) minutes after collection of the filtrate.

At pH 3 the amphipathic structures of flavylium ions are expected to be only abundant anthocyanin forms in the filtrates. These structures contain a full positive charge conferred by the electron-poor oxygen atom in their C (middle) ring. Their self-association is expected to begin upon co-solvation with nicotinamide or nicotinamide riboside in the hot water infusions made by the present invention, and to result in colloidal aggregation processes. The progress of colloidal aggregation in said filtrates was observed as increasing optical depth and viscosity in the filtrates as noted by visual observation after leaving said filtrates standing for eight (8) hours at 20° C. or upon cooling the filtrates to 4° C. for much shorter intervals of less than one hour. Not adding either nicotinamide or nicotinamide riboside to nine grains of pulverized dried calyces of hibiscus sabdariffa added to six (6) ounces of water at 95-100° C. and then infusing and obtaining the filtrate led also to observations of increasing optical depth and increasing viscosity over similar time scales and storing temperatures as used to observe filtrates prepared with added nicotinamide or nicotinamide riboside. Filtrates with or without nicotinamide or nicotinamide riboside were observed to thicken overnight at 20° C.

The filtrates made with the present invention may be consumed soon after preparation in order to obtain effects which are consistent with high nicotinamide or nicotinamide riboside exposure in the hepatic circulation. The effects are marked increases in well-being, energy, and mental focus as noted by the inventor on awakening on the following day after an initial early morning consumption of a filtrate made using the present invention. Consuming the filtrates shortly before bed produced vivid dreams and waking after approximately 4-5 hours of nighttime sleep with extraordinary intellectual activity coupled with a general body relaxation. When the inventor consumed the same quantities of 50 mg nicotinamide or of 125 mg of N-Ribosylnicotinamide chloride with water only, and shortly before bed, then no discernible effects were evident.

An aim of the present invention is to facilitate binding of flavylium colloidal aggregates within the mucosal environment and thereby to increase the numbers of flavylium cations available for uptake across the gastric wall. Also, it is the aim of the present invention to facilitate said binding interactions as needed to retain nicotinamide or nicotinamide ribose with self-associated aggregated forms of flavylium ions and to thereby accomplish their rapid efficient uptake into portal side circulation leading to hepatic metabolism.

It is known to those skilled in the art that increasing cationic charge on particles results in their up-take across cell membranes. Thus, addition of cationic charge to aggregates formed by the present invention (including ones comprising polyphenolics having conjugated pi-electron system) facilitates an uptake process across gastric wall. Cationic anthocyanin (flavylium ion) aggregates in particular are known to rapidly cross the gastric wall to portal side circulation. Localizing cationic charge by complex formation on other molecular structures of the flavonoid class, or on any procyanidins or any proanthocyanidins or any stilbenoids, any of which being obtained from natural, purified or synthetic sources and eventual aggregation of the charged complexes will facilitate binding to gastric wall and eventual uptake of the aggregate forms.

Stability of the aggregates is also facilitated by cross-bridges formed by trivalent (e.g. hibiscus acid or citric acid) anions in solution with cationic charges localized in complexed vitamins, as well as in flavylium structures. This stability particularly favors portal and hepatic cell membranes interactions with complexed vitamins after cell translocation to the portal side.

The present invention taught preparation of nicotinamide and nicotinamide riboside solutions containing their pyridinium cation forms before oral administration. The present invention demonstrated a method of facilitating oral absorption via gastric (stomach) uptake or via intestinal uptake or via both uptake routes into the portal circulation blood for either 50 mg of nicotinamide (3-pyridine-carboxamide) or 110 mg of nicotinamide riboside (from 125 mg of N-ribosylnicotinamide chloride). Other amounts of these vitamins if desired may be used as long as their binding interaction partners on a mole basis remain in excess, and without requiring changes to amounts of the other constituents placed into solutions made as follows. Either vitamin as dry powder of nearly 100% purity was mixed into nine (9) grains of finely-pulverized non-alkalized cocoa powder containing flavan-3-ols, (−) epicatechin, in addition to other flavonols and procyanidins normally present in said cocoa. The amount of said cocoa powder in the mixture may be changed while still serving the aims of the present invention as long as the vitamins are not on a molar basis in excess over their binding partners. In the case of nicotinamide added to said cocoa powder was also added 190 mg of anhydrous citric acid to adjust pH of the resulting solution to approximately pH 3.

Either said vitamin as dry powder of nearly 100% purity was separately mixed into fine powder made of dried calyces of hibiscus sabdariffa flowers (sourced from Monterey Bay Spice Company, Watsonville, Calif. http://www.herbco.com/p-610-hibiscus-flower-powder.aspx containing anthocyanins, proanthocyanidins and hibiscus acid among other constituents normally present in said powder. Separately, either said vitamin was separately mixed into finely-pulverized dried aronia berries (sourced without additives other than aronia berries from http://www.sarenaltd.com, Kyustendil, Bulgaria) Separately, either said vitamin was separately mixed into non-alkalized cocoa powder containing flavan-3-ols, (−) epicatechin, in addition to other flavonols and procyanidins normally present in said cocoa powder (sourced without additives other than cocoa from Fine Cocoa Products Corp. Brooklyn, N.Y. http://www.cocoasupply.com/organic-natural-10-12-cocoa-powder/).

To obtain uniformity in said mixtures of either said vitamin with either said hibiscus, said aronia berry or said cocoa powder, a hi-speed food processor (www.vitamix.com model VM0102D, Cleveland, Ohio) was employed which was also used to process dried whole aronia berries to said finely-pulverized aronia berry. The said aronia berry was also added finely ground powder of anhydrous citric acid in order to adjust pH in water solution to be near pH 3. The said cocoa powder was also added finely ground powder of anhydrous citric acid in order to adjust pH in water solution to below pH 4.1 for mixtures with nicotinamide added. The resulting mixtures obtained with the food processor contained with each nine (9) grains of said aronia berry and 190 mg of said citric acid or contained with each nine (9) grams of said cocoa powder and 190 mg of said citric acid or contained with each nine (9) grams of said cocoa powder and zero (0) mg of said citric acid. The resulting mixtures obtained with the food processor contained with each nine (9) grams of said cocoa powder or with each nine (9) grams of said finely-pulverized aronia berry or with each nine (9) grams of said Hibiscus powder, either 50 mg of nicotinamide or 110 mg of nicotinamide riboside (from 125 mg of N-ribosylnicotinamide chloride). The amount of either vitamin in the mixtures may be changed as long as their binding interaction partners on a mole basis remain in excess, and while still serving the aims of the present invention.

The total number of mixtures was six, two containing said hibiscus sabdariffa, two containing said aronia berries and citric acid, and two containing said cocoa of which one mixture contained citric acid and contained nicotinamide. Each of the mixtures was separately combined with six (6) ounces of either distilled or municipal ‘tap’ water at a temperature just below 100° C. and stirred for 30 seconds. To achieve the aims of the present invention it is unnecessary to prepare solutions with water at a temperature higher than 20° C., although temperature of nearly 100° C. water allowed for much more rapid dissolution of constituents in each mixture versus water at 20° C. Nor is it necessary to add to said mixtures any constituents other than ones indicated here. Any additional constituents to be placed into solution should be evaluated beforehand for possible binding associations that may interfere with or may take the place of any of the said associations that the present invention intends. One mixture at a time was put into solution, stirred, and subsequently consumed by a person in the morning (AM) after rising from sleep. The person swallowed the entire volume of one solution within fifteen (15) minutes after combining each mixture with nearly 100° C. water and refrained from taking additional liquids or eating anything for a minimum of forty-five (45) minutes. A different mixture was placed into water solution each morning such that all the mixtures were consumed.

The solutions made with the present invention may be consumed soon after preparation to obtain in vivo effects consistent with high bioavailability of nicotinamide or nicotinamide riboside or of both vitamins in portal circulation blood and in hepatic tissues. Such effects were noted by the person on awakening the following day after consuming said solutions and included marked increases in well-being, energy, and mental focus. These effects lasted throughout the day and the subsequent evening until sleep time.

In solutions made by the present invention with said hibiscus powder, said cocoa powder, or with said dried aronia berry, solvated anthocyanins or flavan-3-ols will undergo molecular aggregation as a consequence of their amphiphilic (amphipathic) molecular structures. Co-dissolution of pyridinium cations of nicotinamide and nicotinamide riboside in said solutions will result in their cation-π interactions with molecular structures of anthocyanins and flavan-3-ols by virtue of these containing similar prolate arrangement of three rings as a nearly planar pi-electron aromatic system with numbers of aryl hydroxyl groups. Multiple electron donating hydroxyl substituents in said molecular forms favor their participation in cation-π interactions with pyridinium cations of nicotinamide or nicotinamide riboside. Observations of the expected molecular aggregation in said solutions may be made by those skilled in the art after filtration or centrifugation to remove solids by performing (%) transmittance measurements as a function of time. It is an aim of the present invention to facilitate said cation-π interactions in order to form associations of nicotinamide or nicotinamide ribose or both vitamins with aggregating molecular forms and to thereby furnish positively-charged aggregate molecular forms to said mucosal environments in the gastric wall and intestinal wall. It is an aim of the present invention to thereby provide said aggregated forms with bound form of nicotinamide or nicotinamide ribose or of both vitamins to uptake processes within gastric and intestinal cell layers that are responsible for absorption into portal side circulation blood. The pH in the intestinal environment and in portal blood will be expected to result in gradual disappearance of the pyridinium cation form of nicotinamide and consequently the disappearance of cation-π interactions, but need not result in dissociation of bound nicotinamide from the aggregated molecular forms since the pyridine ring of nicotinamide may also participate in other noncovalent interactions within the said aggregate forms. It is also an aim of the present invention to utilize the affinity for hepatic cell membranes of charged or uncharged molecular aggregated forms with bound nicotinamide, bound nicotinamide ribose or with both vitamins bound.

Removal of the hibiscus acid may be done by purification of the anthocyanins from hibiscus sabdariffa or anthocyanins may be purified from another natural source such as from aronia berry or anthocyanidins may be obtained following chemical synthesis and purification. This may be done before using the present invention with the purified anthocyanin or anthocyanidin fractions and may be desirable to limit colloidal aggregate dimensional size after solvation. Alternative counter ions for the flavylium cations may then be introduced which are monovalent, as is the case for chloride ions. Providing a low pH modifier to mixtures of purified anthocyanins or anthocyanidins with nicotinamide or with nicotinamide riboside can be expected by the present invention to obtain binding interactions between the pyridinium nitrogen of either nicotinamide or nicotinamide ribose and aromatic systems of flavylium cations.

The present invention can be obtained by dissolution of nicotinamide or nicotinamide riboside or both vitamins into fluid stomach contents after consuming either vitamin or both vitamins in either a solid or a liquid matrix. In order to obtain availability of their pyridinium cation forms to co-solvation with said aromatic systems contained in molecular structures of any of the major subclasses of flavonoids, said vitamins must enter stomach solutions while said flavonoid molecular structures also undergo solvation in same solution in order to allow said binding interactions which are cation-π binding interactions with the pyridinium cation form(s).

The total number of powder mixtures previously prepared for solutions was six, two of which contained said hibiscus sabdariffa powder, two of which contained said finely-pulverized aronia berries and citric acid, and two of which contained said cocoa powder, one of which contained citric acid when nicotinamide used alone. The mixtures were remade to increase added amounts of nicotinamide and nicotinamide riboside in proportion to the botanical sources of flavonoids. The resulting mixtures obtained with the food processor contained with each six (6) grains of said cocoa powder or with each six (6) grams of said finely-pulverized aronia berry or with each six (6) grams of said Hibiscus powder, either 50 mg of nicotinamide or 110 mg of nicotinamide riboside (from 125 mg of N-ribosylnicotinamide chloride). Each said mixture was put alone into six (6) size 000 gelatin capsules, such that six (6) g of each mixture was transferred into six (6) said capsules.

Each of the mixtures contained in six (6) said capsules was separately swallowed with eight (8) ounces of distilled water at a temperature just below 40° C. over a time period of about 60 seconds. It is unnecessary to add any constituents other than ones indicated here to said mixtures or to water consumed with the mixtures. However, many ingredients may be added without impeding the formation of said pyridinium cations or their interaction with the aromatic systems present in said flavonoid molecular structures. Any additional constituents that may interfere with release of said vitamins or their recognition of said molecular structures in solutions formed within the stomach should be evaluated beforehand. Also, binding associations with the gastric wall constituents represent potential interference with the cation-pi interactions that the present invention intends to engender. Therefore rapid dissolution and mixing in the stomach is highly desirable.

Six (6) gelatin capsules containing one mixture were consumed by a person in the morning (AM) after rising from sleep. The person refrained from taking additional liquids or eating anything for a minimum of forty-five (45) minutes. A different mixture in six capsules was placed into water solution each morning. For example, the nicotinamide containing mixtures were first consumed with hibiscus powder and then with cocoa, followed by nicotinamide riboside containing mixtures with hibiscus and then finally with cocoa.

Effects or benefits obtained using the present invention appear consistent with high bioavailability of nicotinamide or nicotinamide riboside or both vitamins in portal circulation blood and in hepatic tissues. Such effects noted by the inventor included experiencing marked well-being, energy, and mental focus after consuming said mixtures in said gelatin capsules with water. These effects lasted throughout the day and evening until sleep arrived. Particularly of note, were energized and awakened mental states which the inventor experienced upon waking in the morning.

Hibiscus sabdariffa anthocyanins (principally delphinidin 3-O-β-sambubioside and cyanidin-3-O-sambubioside) have molecular structures with a prolate arrangement of three rings and a nearly planar pi-electron aromatic system with aryl hydroxyl groups attached at either extent of the aromatic system. A solution made with said hibiscus sabdariffa powder in distilled water was tested with pH strips and found to have pH value near 3. Using the present invention with said hibiscus powder, flavylium cations are expected to be only abundant anthocyanin forms in solutions that form in the stomach after consuming said gelatin capsules.

It is known that in rodents and humans, ‘anionic’ components situated within the stomach mucosal and intestinal mucosal environments may interact with colloidal protein forms bearing net positive charge. The present invention is to facilitate binding of pyridinium cations of nicotinamide and nicotinamide riboside to said aggregated molecular forms in solutions prepared by the present invention. After consuming said capsules with water, solutions formed in the stomach support appearance of said aggregated forms bearing positive charges and said aggregated forms will demonstrate affinity for anionic components within said mucosal environments. It is known that flavonoids that are anthocyanins undergo efficient and rapid absorption from the stomach of rats and humans, including cases where water infusions made from dried calyces of hibiscus sabdariffa were consumed orally by human subjects followed by pharmacokinetic observations of efficient in vivo plasma exposure to hibiscus anthocyanins.

The present invention may be envisioned to be used with any of the major subclasses of flavonoids, anthocyanidins (anthocyanins), flavan-3-ols, flavonols, flavanones, flavones, and isoflavones, or any procyanidins or any proanthocyanidins or any stilbenoids, any of which being obtained from natural, purified or synthetic sources. Choosing among these may be designed to limit colloidal aggregated sizes to ranges suitable for gastric and intestinal absorption of the aggregated forms. The present invention controls solution pH to below pH 4.1, preferably near or below pH 3 in order to form an abundance of pyridinium cation of nicotinamide. Control over solution pH in same range is desirable also with nicotinamide riboside, if anthocyanins are selected for using present invention, as this pH range ensures predominantly flavylium cations are present in solutions produced by the present invention.

In summary, the present invention provides a novel approach to improve absorption of nicotinamide or nicotinamide riboside in a subject including methods enabling high concentrations of either or both vitamins to reach enzymes in portal and hepatic system. Thereby driving biotransformation and increasing their distribution as metabolized forms. Although the potential methods and applications of using the same according to the present invention have been described in the foregoing specification with considerable details, it is to be understood that modifications may be made to the invention which do not exceed the claimed scope of the invention and modified forms of the present invention done by others skilled in the art to which the invention pertains will be considered infringements of this invention when those modified forms fall within the claimed scope of this invention.

What is claimed is:

1. A method to improve oral absorption of nicotinamide or nicotinamide riboside or both nicotinamide and nicotinamide riboside via gastric (stomach) uptake or via intestinal uptake into the portal circulation blood by mixing nicotinamide or nicotinamide riboside or both nicotinamide and nicotinamide riboside with anthocyanin(s) or flavan-3-ol(s) of flavonoids to form positively-charged aggregating molecular forms through co-solvation of flavan-3-ols or anthocyanins or the flavylium ion of anthocyanins with the pyridinium cation form of nicotinamide or nicotinamide riboside or with the pyridinium cation forms of nicotinamide and nicotinamide riboside, wherein said anthocyanin(s) or flavan-3-ol(s) remains in excess of nicotinamide or nicotinamide riboside or both nicotinamide and nicotinamide riboside on a mole basis.
Wherein said nicotinamide as 3-pyridine-carboxamide or said nicotinamide riboside as N-ribosylnicotinamide chloride or both nicotinamide and nicotinamide riboside as dry powder of nearly 100% purity is mixed with finely-pulverized dried calyces of hibiscus sabdariffa flowers to form a mixture for co-solvation by water or in a solid powder form to form a mixture contained in gelatin capsules for co-solvation by water after oral administration.
Wherein said nicotinamide as 3-pyridine-carboxamide or both nicotinamide and nicotinamide riboside as dry powder of nearly 100% purity is mixed with cocoa powder to form a mixture for co-solvation by water or in a solid powder form to form a mixture contained in gelatin capsules where citric acid or gluconic acid or another pH modifier is added to adjust pH value to be below 4.1 for co-solvation by water after oral administration, wherein said nicotinamide riboside as N-ribosylnicotinamide chloride as dry powder of nearly 100% purity is mixed with cocoa powder to form a mixture for co-solvation by water or in a solid powder form to form a mixture contained in gelatin capsules where citric acid or gluconic acid or another pH modifier is not required for co-solvation by water after oral administration.
Wherein said nicotinamide as 3-pyridine-carboxamide or both nicotinamide and nicotinamide riboside as dry powder of nearly 100% purity is mixed with finely-pulverized aronia berries to form a mixture for co-solvation by water or in a solid powder form to form a mixture contained in gelatin capsules where citric acid or gluconic acid or another pH modifier is added to adjust pH value to be below 4.1 for water solvation, wherein said nicotinamide riboside as N-ribosylnicotinamide chloride as dry powder of nearly 100% purity is mixed with finely-pulverized aronia berries to form a mixture for co-solvation by water or in a solid powder form to form a mixture contained in gelatin capsules where citric acid or gluconic acid or another pH modifier is preferred to adjust pH value to be below 4.1 for co-solvation but not required.

2. The method to improve oral absorption of nicotinamide or nicotinamide riboside or both nicotinamide and nicotinamide riboside in claim 1, wherein said mixture in water solution is consumed by a person in the morning time after rising from sleep before taking any additional liquid or food, wherein said mixture in solid powder form in gelatin capsules is consumed by a person with water in the morning time after rising from sleep before taking any additional liquid or food.
